(19) United States
(12) Patent Application Publication
(10) Pub. No.: US 2024/0019845 A1
(43) Pub. Date: Jan. 18, 2024

(54) HIGH THROUGHPUT SCREENING

(71) Applicants: BASF CORPORATION, Florham Park, NJ (US); BASF SE, Ludwigshafen am Rhein (DE)

(72) Inventors: Brian Yoo, White Plains, NY (US); Kevin JABLONKA, Sion (CH)

(21) Appl. No.: 18/034,932

(22) PCT Filed: Nov. 11, 2021
(86) PCT No.: PCT/EP2021/080426

§ 371 Date: May 2, 2023

Related U.S. Application Data
(60) Provisional application No. 63/108,768, filed on Nov. 2, 2020.

(51) Int. Cl. G05B 19/4155

(52) U.S. Cl. CPC G05B 19/4155; G05B 2219/32168

(57) ABSTRACT

An apparatus for controlling synthesis of a material in particular a polymer is proposed, the apparatus comprising at least: an obtaining unit configured to receive a digital representa-tion of a candidate material, a model unit configured to provide a data driven model trained based on digital representations of previously presented materials and at least two of their respective characteristic properties, a pareto unit configured to provide a provisional pareto front associated with the at least two characteristic material properties for the subset of materials, a property determination unit configured to determine the at least two character-istic material properties of the candidate material based on the data driven model and the digital representation, a validation unit configured to compare the determined at least two characteristic material properties with the provisional pareto front, a providing unit config-ured to, based on the Ccomparison providing a control file, suitable for controlling the syn-thesis of the candidate material.

### TECHNICAL FIELD

The present disclosure relates to a method, a system and a computer program product for determining a provisional pareto front of characteristic properties of materials for screening. The current disclosure further relates to an apparatus, a method and a computer program product for controlling synthesis of a material in particular a polymer.

### TECHNICAL BACKGROUND

In chemical industries development of new materials based on technical application requirement is a key target. This is in particular challenging, when developing new polymers. A common problem is to identify an optimal material for a given application. An optimal material is generally defined by its property, more specifically by its technical application property or in other words by its respective characteristic property. For most practical applications, several properties, and not one single objective, are relevant. Finding an optimal material, that fulfills target requirements is difficult. Thus, there is a need for an improved way of identifying optimal materials.

SUMMARY OF THE INVENTION

On one aspect an apparatus for controlling synthesis of a material in particular a polymer is proposed, the apparatus comprising at least:
an obtaining unit configured to receive a digital representation of a candidate material,
a model unit configured to provide a data driven model trained based on digital representations of previously presented materials and at least two of their respective characteristic properties,
a pareto unit configured to provide a provisional pareto front associated with the at least two characteristic material properties for the subset of materials,
a property determination unit configured to determine the at least two characteristic material properties of the candidate material based on the data driven model and the digital representation,
a validation unit configured to compare the determined at least two characteristic material properties with the provisional pareto front,
a providing unit configured to, based on the comparison providing a control file, suitable for controlling the synthesis of the candidate material.

In another aspect a computer implemented method for controlling synthesis of a material, in particular a polymer is proposed, the method comprising the steps of: A computer implemented method for controlling synthesis of a material, in particular a polymer is proposed, the method comprising the steps of:
providing a digital representation associated with a synthesis specification of a candidate material
providing a data driven model trained based on digital representations of previously presented materials and at least two of their respective characteristic properties,
determining the at least two characteristic material properties of the candidate material based on the data driven model and the digital representation,
comparing the determined at least two characteristic material properties with the provisional pareto front,
based on the comparison providing a control file, suitable for controlling the synthesis of the candidate material.

In another aspect computer program product for controlling synthesis of a material, in particular a polymer is proposed, the computer program product comprising instructions, which, when executed on computing devices of a computing environment, is configured to carry out the steps of the method of controlling synthesis of a material.

In another aspect a non-transitory computer-readable storage medium, the computer-readable storage medium including instructions that when executed by a computer, cause the computer to the steps of the method of controlling synthesis of a material. In an aspect use of the provided control file for controlling synthesis of the candidate material is proposed.

In another aspect, a computing apparatus for determining a provisional pareto front of characteristic material properties of materials in particular for screening is proposed, the apparatus comprising
a processing device, and
a memory storing instructions that, when executed by the processor, configure the apparatus to perform the steps of
provide via a communication interface a set of materials, wherein each of the materials of the set of materials is described by their digital representation
provide via the communication interface for of each of the materials of a subset of the set of materials at least two of their respective characteristic properties;
provide a data driven model trained based on the digital representation of each of the materials of the subset of materials and at least two of their respective characteristic properties;
predict with the processing device the characteristic material properties of remaining materials from the set of materials based on the data driven model;
provide via the communication interface for each of the set of materials their characteristic material properties;
determine with the processing device from the set of materials a predicted pareto optimum for the at least two of the characteristic material properties;
provide via the communication interface the determined provisional pareto front.

In another aspect, a computer implemented method for determining a provisional pareto front of characteristic material properties of materials in particular for screening is proposed, the method comprising the steps of:
providing via a communication interface a set of materials, wherein each of the materials of the set of materials is described by their digital representation
providing via the communication interface for of each of the materials of a subset of the set of materials at least two of their respective characteristic properties,
providing a data driven model trained based on the digital representation of each of the materials of the subset of materials and at least two of their respective characteristic properties;
predicting with the processing device the at least two characteristic material properties of remaining materials from the set of materials based on the data driven model;
providing via the communication interface for each of the set of materials their at least two characteristic material properties,
determining with the processing device from the set of materials a predicted pareto optimum for the at least two of the characteristic material properties,
providing via the communication interface the determined provisional pareto front.

In another aspect computer program product for determining a provisional pareto front of characteristic material properties of materials in particular for screening is proposed, the computer program product comprising, instructions that when executed by a processing device perform the steps of the method for determining a provisional pareto front.

In another aspect a non-transitory computer-readable storage medium, the computer-readable storage medium including instructions that when executed by a computer, cause the computer to the steps of the method for determining a 5.

Any disclosure and embodiments described herein relate to the methods, the systems, devices, the computer program product lined out above and vice versa. Advantageously, the benefits provided by any of the embodiments and examples equally apply to all other embodiments and examples and vice versa.

The methods, apparatuses, computer program products and computer readable media disclosed herein provide an efficient way of finding new materials with optimal or at least improved technical application properties. Due to the large experimental space it is combinatorically prohibitive to exhaustively enumerate or to experimentally access the experimental space for finding optimal or improved materials. In particular, when more than one technical application property is desired, the experimental space is large. By use of the invention it is possible to quickly identify promising material candidates. In case of controlling the experiment, the resources for performing experiments can be reduced. This is enabled by comparing and then providing the control file based on the comparing step.

In an aspect a computer implemented method for reducing the design space is for material development is proposed, comprising the steps of
providing via a communication interface a set of materials, wherein each of the materials of the set of materials is described by their digital representation,
providing via a communication interface at least two characteristic material properties for each material of the set of materials, comprising providing via the communication interface for of each of the materials of a subset of the set of materials their respective characteristic properties and providing the predicted characteristic material properties for each of the remaining set of materials classifying with a processing device. The method may further comprise the step of training with the processing device a data driven model, based on the digital representation of each of the materials of the subset of materials and their respective characteristic properties.

As used herein “determining” also includes “initiating or causing to determine”, “generating” also includes “initiating or causing to generate” and “providing” also includes “initiating or causing to determine, generate, select, send or receive”. “Initiating or causing to perform an action” includes any processing signal that triggers a computing device to perform the respective action. Determining may further include predicting based on a data driven model.

“Pareto front” refers to the set of pareto-optimal solutions. Pareto optimal is a situation where no individual or preference criterion can be improved without penalizing at least one different individual or preference criterion. A provisional pareto front may refer to a pareto front that is an approximation of an exact pareto front, wherein the provisional pareto front comprises pareto dominant materials.

“Materials” may refer to a chemical substance or mixture of substances, a chemical substance may be e. g. polymers, emollients, formulations, mixtures, alloys, ceramics, glasses.

“Polymer” refers to a substance or material comprising large molecules composed of repeating subunits, examples of these subunits may be monomers.

“Test candidate” may refer to a material proposed for sampling. Sampling may refer to experiments either in a laboratory or in silico.

“Pareto dominant” may refer to a situation, when for at least one of the at least two of the characteristic material properties the material is superior to other materials and at the same time is not inferior in any of the at least two characteristic properties.

“Characteristic material properties” may refer to physical or chemical properties of a material, in particular to technical application properties of the material. Characteristic material properties may relate to molecular properties. The characteristic material properties may also be named objectives.

“Communication interface” may comprise a physical interface (e.g. a keyboard, a touch screen, a computer mouse, etc.) The communication interface may comprise a logical interface (e. g. computer interface to a database, a wired or wireless interface to a computer or a computer network, API, etc.). In another aspect the communication interface may be one interface or several interfaces. In particular, each determination step may be performed at a separate processor, implying that for each providing step a separate interface may be used. The communication interface may further refer to a display device.

“Digital representation” may refer to a representation of a material. In particular this may be a structural formula, a brand name, a CAS number, a formulation, SMILES representation, the digital representation may be associated with a synthesis specification.

“Synthesis specification” may refer to a recipe for synthesizing a material, in particular a polymer and may comprise a digital representation of ingredients needed for the synthesis and instructions for synthesizing. The synthesis specification may be provided in form of a control file.

“Data driven model” may refer to a model at least partially derived from data. The data driven model may be a data driven model for predicting technical application properties, the data driven model may comprise separate data driven models in particular one for each technical application property. Use of a data driven model can allow describing relations, that cannot be modelled by physico-chemical laws. The use of data driven models can allow to describe relations without solving equations from physico-chemical laws. This can reduce computational power. This can improve speed. The data driven model may be derived from statistics (Statistics 4th edition, David Freedman et al., W. W. Norton & Company Inc., 2004). The data driven model may be derived from Machine Learning (Machine Learning and Deep Learning frameworks and libraries for large-scale data mining: a survey, Artificial Intelligence Review 52, 77-124 (2019), Springer). The data driven model may comprise black xo models,

“Black box model” may refer to models be built by using one or more of Machine Learning, deep learning, neural networks, or other form of artificial intelligence. The black-box-model may be any model that yields a good fit between training and test data.

The data driven model may comprise a white box model.

“White box model” refers to models based on physico-chemical laws. The physico-chemical laws may be derived from first principles. The physico-chemical laws may comprise one or more of chemical kinetics, conservation laws of mass, momentum and energy, particle population in arbitrary dimension. The white-box-model may be selected according to the physico-chemical laws that govern the respective problem.

The data driven model may comprise hybrid models.

“Hybrid model” may refer to a model that comprises white box models , black box models , see e.g. review paper of Von Stoch et al., 2014, Computers & Chemical Engineering, 60, Pages 86 to 101. The trained model may comprise a combination of a white-box-model and a black-box-model.

“Machine Learning” may refer to computer algorithms that improve through experience, Machine Learning algorithms build a model based on sample data, often described as training data. “Processing device” may be a computer or even a general-purpose processing device such as a microprocessor, microcontroller, central processing unit (“CPU”), or the like. More particularly, the processing device may be a CISC (Complex Instruction Set Computing) microprocessor, RISC (Reduced Instruction Set Computing) microprocessor, VLIW (Very Long Instruction Word) microprocessor, or a processor implementing other instruction sets or processors implementing a combination of instruction sets. The processing device or processing means may also be one or more special-purpose processing devices such as an ASIC (Application-Specific Integrated Circuit), an FPGA (Field Programmable Gate Array), a CPLD (Complex Programmable Logic Device), a DSP (Digital Signal Processor), a network processor, or the like. The methods, systems and devices described herein may be implemented as software in a DSP, in a micro-controller, or in any other side-processor or as hardware circuit within an ASIC, CPLD, or FPGA. As outlined also earlier, it is to be understood that the term “processing device” or processor may also refer to one or more processing devices, such as a distributed system of processing devices located across multiple computer systems (e.g., cloud computing), and is not limited to a single device unless otherwise specified. Moreover, any one or more of the processing devices may be located at a physical location which is different from the other processing devices.

“Remaining materials” may refer to materials where the characteristic material properties are not yet determined by simulation and/or experiments.

In an embodiment the synthesis specification of the candidate material comprises a list of ingredients and machine-readable instructions for synthesizing material.

This allows controlling synthesis in a fully automated laboratory, which greatly increases throughput in the laboratories.

In an embodiment, the digital representation of a candidate material may be provided by a client device and the control file may be received by a client device. This adds flexibility, the client device may be located in a separate location from the data processing location. The method according to any one of the preceding claims, wherein the provisional pareto front comprises a measure for uncertainty. The measure for uncertainty in the provisional pareto front allows early use of the method although the provisional pareto front still has a large uncertainty. This allows to include materials, where the material characteristics are only determined with a larger uncertainty. This reduces the burden of experiments and or simulations.

In an aspect, the provisional pareto front may comprise the materials classified as pareto dominant. The additional classifier allows easy reconstruction and amendment of the provisional pareto front. This enhances speed of providing the provisional pareto front.

In an embodiment the at least two determined characteristic properties comprise a measure for uncertainty. Including the uncertainty of the at least two determined characteristic properties accounts for experimental errors or uncertainties of the data driven model. This enables to find candidate materials as pareto optimal although the data driven model provides an average that would not overlap with the provisional pareto front. Consequently, this enables more accurate results in determining material with pareto optimal properties. And reduces the danger of neglecting candidate materials with optimal characteristic properties.

In an embodiment the step of comparing comprises determining if the determined characteristic material properties overlap with the provisional pareto front. Overlap may mean that the least two characteristic properties exceed the provisional pareto front. Overlapping of the determined with the provisional pareto front is easy to determine and thereby reduces calculation times and improves speed.

In an embodiment the control file is provided if the comparing step determines an overlap. This reduces the resources as synthesis is only initiated for candidate materials that are likely to provide an improvement.

In an embodiment a step of controlling the synthesis is comprised, in particular based on the provided control file, more particular by controlling flow rates of ingredients and reaction temperatures. Controlling of the synthesis allows optimal use of the synthesis equipment. A single operator may supervise several synthesis equipment simultaneously by reducing manual control.

In an embodiment, a step of training with the processing device the data driven model, based on the digital representation of each of the materials of the subset of materials and their respective characteristic properties. The subset of materials may be understood as the previously presented materials.

In an embodiment providing via the communication interface the provisional pareto front comprises providing the trained data driven model.

In an embodiment, providing via the communication interface the characteristic material properties for each of the set of materials may comprise providing via the communication interface for of each of the materials of the subset of the set of materials their respective characteristic properties and providing the predicted characteristic material properties for each of the remaining set of materials.

In an embodiment, determining the provisional pareto front may comprise classifying materials as pareto dominant.

In an embodiment, determining the provisional pareto front may comprise classifying materials as pareto dominated. “Pareto dominated” may refer to a situation, where at least one other material is pareto dominant.

Classifying each material of the subset of materials or in other words the previously presented materials is a simple and computational cheap approach of identifying pareto optimal materials and thereby a provisional pareto front. Furthermore, classifying based on the characteristic properties allows an easy interpretation of the resulting provisional pareto front. This avoids the need to define a new performance indicator by combining the characteristic properties in an arbitrary way, which is likely biased. Furthermore, the new performance indicator will be difficult to interpret. While defining a new performance indicator by combining the characteristic properties in an arbitrary way may still be feasible it becomes computationally more challenging with an increasing number of characteristic properties. In addition, for each combination of characteristic properties a new performance indicator has to be created and the provisional pareto front needs to be determined.

In an embodiment, the provisional pareto front may be defined by the materials classified as pareto dominant. This allows easy retrieval of the provisional pareto front. A database comprising previously materials and their respective at least two characteristic properties can easily be browsed for the classifier and reconstruct the provisional pareto front accordingly. This enables a fast way of providing a provisional pareto front and also allows easy update of the provisional pareto front. This enables high accuracy and reliability of the provisional pareto front.

In an embodiment, materials being neither pareto dominant nor pareto dominated may remain unclassified. This allows easy identification of materials that may be candidate materials.

In an embodiment, the materials classified as pareto dominated may be discarded. Discarded materials will not be synthesized. This reduces the workload on the synthesis equipment.

In an aspect the provisional pareto front may comprise the undiscarded materials. A way of classifying pareto dominants may be performed as described in Zuluaga, M.; Krause, A.; Puschel, M. e-PAL: An Active Learning Approach to the Multi-Objective Optimization Problem. J. Mach. Learn. Res. 2016,17,1-32 or Zuluaga, M.; Sergent, G.; Krause, A.; Puschel, M. Active Learning for Multi-Objective Optimization. Proceedings of the 30th International Conference on Machine Learning, Atlanta, Georgia, USA, 2013; pp 462-470. In an aspect, the provisional pareto front may comprise the materials classified as pareto dominant.

In an embodiment, the at least two characteristic material properties of each of the materials may comprise uncertainty estimates. Hence, the provisional pareto front may comprise an uncertainty estimate in other words measure for uncertainty. One example the measure for uncertainty may be a lower and an upper limit of the provisional pareto front, e. g. defined by uncertainties of the pareto dominant classified materials.

The uncertainty estimate may be defined by errors in determining characteristic material properties by simulations and/or measurements. An uncertainty estimate may be defined by uncertainties in predicting characteristic material properties using the trained data driven model. These uncertainty estimates may form hyperrectangles in the space of characteristic material properties. In more mathematical terms the characteristic material properties are often referred to as objectives. Another term for characteristic material properties may be performance indicator. Throughout this disclosure the terms objective, characteristic material properties and performance indicator, technical application property may be used synonymously.

In an embodiment the at least two characteristic material properties of each of the materials may comprise uncertainty estimates.

In an embodiment, the determined at least two characteristic material properties of remaining material(s) from the set of materials based on the data driven model comprise uncertainty estimates.

In an embodiment, providing via the communication interface the characteristic material properties for each material of the set of materials may comprise providing the uncertainty estimate for each of the characteristic material properties for each material of the set of materials. In other words providing the hyperrectangle for each of the materials. The use of a hyperrectangle is easily interpretable and allows easy visual inspection of the results.

In an embodiment, providing the uncertainty estimate for each of the characteristic material properties for each material of the set of materials comprises providing the uncertainty estimate defined by errors in determining characteristic material properties by simulations and/or measurements, for each of the materials when the uncertainty estimates defined by errors in determining characteristic material properties by simulations and/or measurements are available.

The characteristic material properties determined by simulations and/or measurements are generally more accurate than the predicted characteristic material properties. This leads to a reduced uncertainty estimate. Consequently, the classification will be more accurate. In an embodiment, classifying materials as pareto dominant, e. g. when for at least one of the at least two of the characteristic properties the material is superior to other materials by at least a margin ε and at the same time is not inferior in any of the at least two characteristic properties. This provides a clear classification rule, that is interpretable as well as easy to determine.

In an embodiment, determining the provisional pareto front may comprise classifying materials as pareto dominated e. g. when at least one other material is pareto dominant by at least a margin ε.

In an embodiment the method may comprise ranking unclassified materials based on the magnitude of their respective uncertainty estimates. Ranking allows batch processing in a given order. This is in particular useful, when digital representation of more than one candidate material are provided. Furthermore, it allows a better use of the resources of the synthesis equipment.

In an embodiment the method may comprise ranking the undiscarded materials based on the magnitude of their respective uncertainty estimates. Ranking allows batch processing in a given order. This is in particular useful, when digital representation of more than one candidate material are provided. Furthermore, it allows a better use of the resources of the synthesis equipment.

In an embodiment the ranking may be prioritized by weighting characteristic material properties.

In an embodiment, the method may comprise providing via the communication interface a proposed material or a batched of materials for sampling.

In an embodiment the proposed material for sampling may comprise the highest ranked unclassified material or a batch of unclassified materials.

In an embodiment the proposed material for sampling may comprise the highest ranked undsicarded material or a batch of undiscarded materials.

In an embodiment the proposed material for sampling may be sampled, thereby determining the characteristic material properties of the proposed material.

In an embodiment, the method may further comprise providing the determined characteristic material properties of the sampled material or the batch of material.

In an embodiment the method my further comprise providing determined characteristic material properties of the proposed material or a batch of materials.

In an aspect the method may further comprise retraining the data driven model based on the proposed material and the determined the characteristic material properties of the proposed material or batch of material.

Retraining the data driven model increases the prediction accuracy of the data driven model. This decreases the uncertainty estimate for the predicted characteristic material properties.

In an embodiment, providing the trained model comprises providing the retrained model.

In an embodiment, a feature set is derived from the digital representation.

According to an aspect, a computer program or a computer program product or computer readable non-volatile storage medium comprising computer readable instructions, which when loaded and executed by a processing device perform the methods disclosed herein.

According to an aspect a system is proposed, the system comprising an input device, and output device and a processing device configured for performing the method disclosed herein.

The disclosure applies to the systems, methods, computer programs, computer readable non-volatile storage media, computer program products disclosed herein alike. Therefore, no differentiation is made between systems, methods, computer programs, computer readable non-volatile storage media or computer program products. All features are disclosed in connection with the systems, methods, computer programs, computer readable non-volatile storage media, and computer program products disclosed herein.

An exemplary implementation is provided as a computer implemented method for determining a provisional pareto front of characteristic material properties of materials in particular for screening comprising the steps of
providing via a communication interface a set of materials, wherein each of the ma20 terials of the set of materials is described by their digital representation
providing via the communication interface for of each of the materials of a subset of the set of materials at least two of their respective characteristic properties;
providing a data driven model trained based on the digital representation of each of the materials of the subset of materials and at least two of their respective characteristic properties;
predicting with the processing device the characteristic material properties of remaining materials from the set of materials based on the data driven model;
providing via the communication interface for each of the set of materials their characteristic material properties;
determining with the processing device from the set of materials a predicted pareto optimum for the at least two of the characteristic material properties;
providing via the communication interface the determined provisional pareto front,
wherein the at least two characteristic material properties of each of the materials comprises uncertainty estimates,
wherein providing via the communication interface the characteristic material properties for each material of the set of materials may comprise providing the uncertainty estimate for each of the characteristic material properties for each material of the set of materials, wherein providing the uncertainty estimate for each of the characteristic material properties for each material of the set of materials comprises
providing the uncertainty estimate defined by errors in determining characteristic material properties by simulations and/or measurements, for each of the materials when the uncertainty estimates defined by errors in determining characteristic material properties by simulations and/or measurements are available wherein classifying materials as pareto dominant, comprises
classifying a material as pareto dominant when for at least one of the at least two of the characteristic properties the material is superior to other materials by at least a margin ε and at the same time is not inferior in any of the at least two characteristic properties, wherein classifying materials as pareto dominated comprises in classifying materials as pareto dominated when at least one other material is pareto dominant by at least a margin ε, comprising
ranking unclassified materials based on the magnitude of their respective uncertainty estimates
providing via the communication interface a proposed material for sampling or a batch of materials for sampling based on the ranking
providing determined characteristic material properties of the proposed material or a batch of materials
retraining the model based on the proposed material and the determined the characteristic material properties of the proposed material or batch of materials
providing the trained model comprises providing the retrained model.

### BRIEF DESCRIPTION OF THE DRAWINGS

To easily identify the discussion of any particular element or act, the most significant digit or digits in a reference number refer to the figure number in which that element is first in30 troduced.

FIG. 1 illustrates a routine 100 for determining a provisional pareto front of characteristic material properties of materials in particular for screening in accordance with one embodiment.

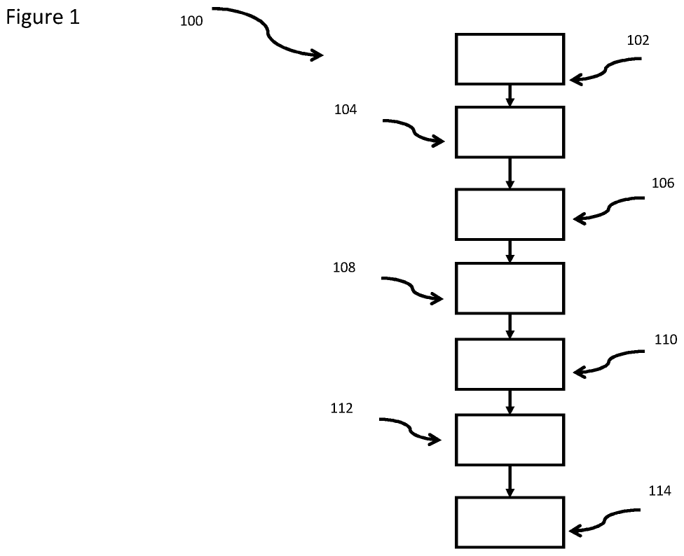

FIG. 2 illustrates a routine 200 for determining a provisional pareto front of characteristic material properties of materials in particular for screening in accordance with one embodiment.

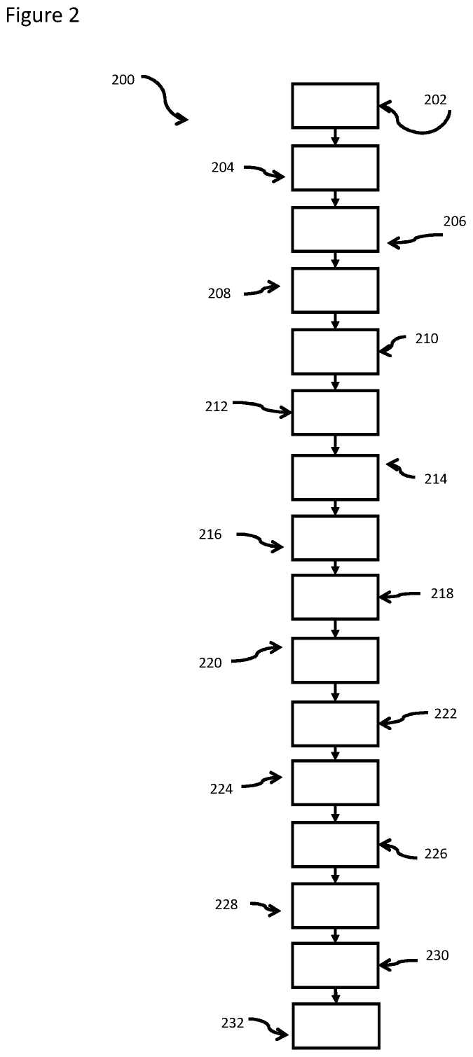

FIG. 3 illustrates an aspect of the subject matter in accordance with one embodiment.

FIG. 4 illustrates an implementation of the method of controlling synthesis of a material

FIG. 5 illustrates an example of an apparatus for controlling synthesis of a material

### DETAILED DESCRIPTION

In block 102, routine 100 provides via a communication interface a set of materials, wherein each of the materials of the set of materials is described by their digital representation. In this example the set of materials is a design space. In this example the design space considered of polymers was evaluated. For the design space, four monomer types and chain lengths between sixteen and forty-eight in increments of two were considered. It was further considered that the reverse sequence equals the forward sequence. The total number of polymers in the design space may then be determined. This results in more than 14 million possible sequences. Enumeration is impossible for so many polymers. For example, assuming an average memory requirement of 62 kB per simplified molecular-input lineentry system (SMILES) the memory footprint would correspond to 0.8 TB. In this example the design space has been limited by design of experiment (DOE). Therefore, the set of materials is a reduction from the full design space. Reducing the design space using DOE may be an optional step.

In block 104, routine 100 provides via the communication interface for of each of the materials of a subset of the set of materials at least two of their respective characteristic properties.

In this example, the characteristic material properties are the adsorption free energy, the dimer free energy barrier and the radius of gyration. For other materials different characteristic material properties may be used. The characteristic material properties in this example, where determined by simulations in a step previous before the step providing (104). In other examples the characteristic material properties may be determined by experiment. In further examples, the characteristic material properties may be determined by simulation and experiments.

In block 106, routine 100 provides a data driven model trained based on the digital representation of each of the materials of the subset of materials and at least two of their respective characteristic properties. In block 108, routine 100 predicts with the processing device the at least two characteristic material properties of remaining materials from the set of materials based on the data driven model. In block 110, routine 100 provides via the communication interface for each of the set of materials their at least two characteristic material properties. In block 112, routine 100 determines with the processing device from the set of materials a predicted pareto optimum for the at least two of the characteristic material properties. In block 114, routine 100 provides via the communication interface the determined provisional pareto front.

In block 202, routine 200 provides via a communication interface a set of materials, wherein each of the materials of the set of materials is described by their digital representation. In block 204, routine 200 provides via the communication interface for of each of the materials of a subset of the set of materials at least two of their respective characteristic properties.

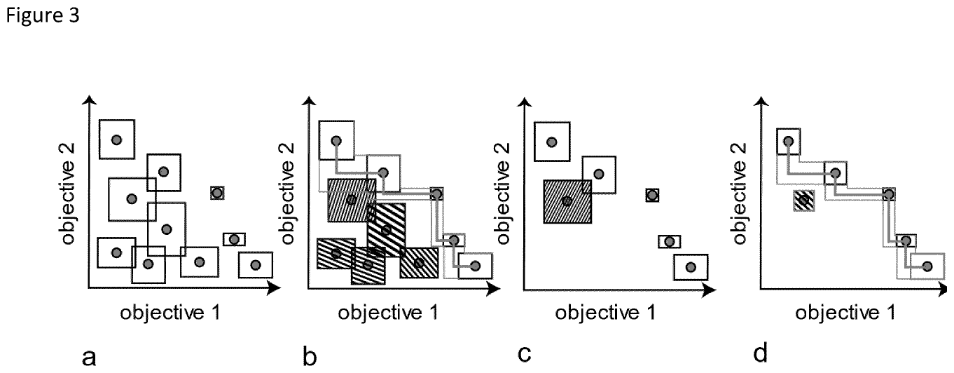

In block 206, routine 200 provides a data driven model trained based on the digital representation of each of the materials of the subset of materials and at least two of their respective characteristic properties. In block 208, routine 200 predicts with the processing device the characteristic material properties of remaining materials from the set of materials based on the data driven model. In block 210, routine 200 provides via the communication interface for each of the set of materials their characteristic material properties. In block 212, routine 200 determines with the processing device from the set of materials a predicted pareto optimum for the at least two of the characteristic material properties. In block 214, routine 200 provides via the communication interface the determined provisional pareto front. In block 216, routine 200 wherein the at least two characteristic material properties of each of the materials comprises uncertainty estimates. In block 218, routine 200 wherein providing via the communication interface the characteristic material properties for each material of the set of materials may comprise providing the uncertainty estimate for each of the characteristic material properties for each material of the set of materials, wherein providing the uncertainty estimate for each of the characteristic material properties for each material of the set of materials comprises. In block 220, routine 200 provides the uncertainty estimate defined by errors in determining characteristic material properties by simulations and/or measurements, for each of the materials when the uncertainty estimates defined by errors in determining characteristic material properties by simulations and/or measurements are available wherein classifying materials as pareto dominant, comprises. In block 222, routine 200 classifies a material as pareto dominant when for at least one of the at least two of the characteristic properties the material is superior to other materials by at least a margin ε and at the same time is not inferior in any of the at least two characteristic properties, wherein classifying materials as pareto dominated comprises In classifying materials as pareto dominated when at least one other material is pareto dominant by at least a margin ε, comprising. In block 224, routine 200 ranking unclassified materials based on the magnitude of their respective uncertainty estimates. In block 226, routine 200 provides via the communication interface a proposed material for sampling or a batch of materials for sampling based on the ranking. In block 228, routine 200 provides determined characteristic material properties of the proposed material or a batch of materials. In block 230, routine 200 retrains the model based on the proposed material and the determined the characteristic material properties of the proposed material or batch of materials. In block 232, routine 200 provides the trained model comprises providing the retrained model. In FIG. 3 a schematic flow of determining the provisional pareto front is shown. FIG. 3 a) each black dot depicts a characteristic material property 302 of a material in the material property space. In this non limiting example the material property space is 2-d, one property dimension is depicted as objective 1, the other property dimension is depicted as objective 2. Each characteristic material property 302 is surrounded by their respective uncertainty estimates 304. In FIG. 3 b pareto dominated materials 306 are depicted with hashed uncertainty estimates 312, pareto dominating materials 308 are classified, and belong to the provisional pareto front 310. Materials that are neither pareto dominant nor pareto dominated are unclassified material 314. In FIG. 3 c the pareto dominated materials 306 have been discarded. Only one unclassified material 314 is present and is proposed for sampiing. After sampling, the uncertainty estimates 304 of the sampled material are reduced in size as the error of the sampling is typically smaller than the error of the prediction model. FIG. 3 d shows the pareto dominant values and the previously unclassified material 314 after retraining of the data driven model. It can be seen that the uncertainty estimates 304 is reduced for various pareto dominant materials. This is, because the added training datum of the freshly sampled unclassified material 314 increases performance of the prediction model. It is clearly visible that unclassified material 314 now belongs to the class of pareto dominated materials and can be discarded.

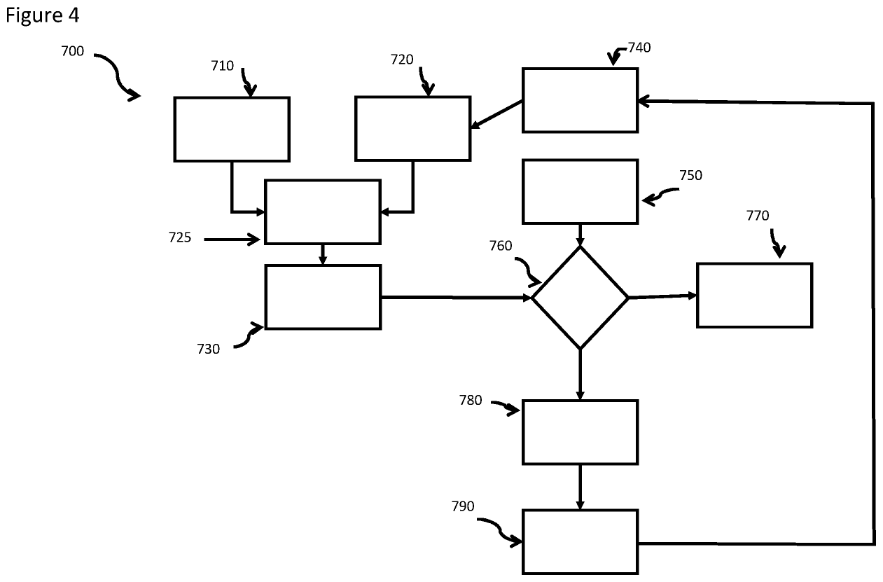

In FIG. 4, an exemplary implementation of a method (700) of controlling synthesis of a specification is shown. At step 700 a digital representation associated with a synthesis specification of a candidate material is provided, in this example, the candidate material is a candidate polymer, and the digital representation associated with the synthesis specification. In other examples, the synthesis specification may be derived from a database. At step 710, a data driven model is provided, the data driven model is trained based on digital representations of previously presented materials and at least two of their respective characteristic properties. at steps 725 the at least two characteristic material properties of the candidate material based on the data driven model and the digital representation are determined. At an optional step 730 They determined at least two characteristic material properties may be provided. At step 750 the provisional pareto front is provided. In the next step 760 the determined at least two characteristic material properties are compared with the provisional pareto front. Based on the comparison step 760 a controlled signal suitable for controlling the synthesis of the material is provided. the comparison step determines whether the candidate material will improve the provisional pareto front or not. If no improvement is to be expected the method step 770 will discard synthesis of the material and the controlled signal may indicate that no synthesis needs to be performed, by sending a stop signal to the synthesis apparatus. An improvement is to be expected when the provisional pareto front and they determined at least two characteristic material properties overlap. In that case at step 780 the control file initiating synthesis of the material will be provided, and synthesis of the candidate material is initiated. The control file may comprise a list of ingredients for synthesizing the material and instructions for synthesis. At step 790 experiments are initiated that measure the at least two characteristic material properties of the candidate material. The results from these measurements may then be provided to update the data driven model at step 740. Updating the data driven model reduces uncertainties for the material characteristics. Updating of the data driven model may be performed by retraining the data driven model with the additional data. In an optional step, the measured characteristics of the material property of the candidate may be compared to the provisional pareto front and the provisional pareto front may be updated. E. g. by classifying the material characteristics of the candidate material as pareto dominant.

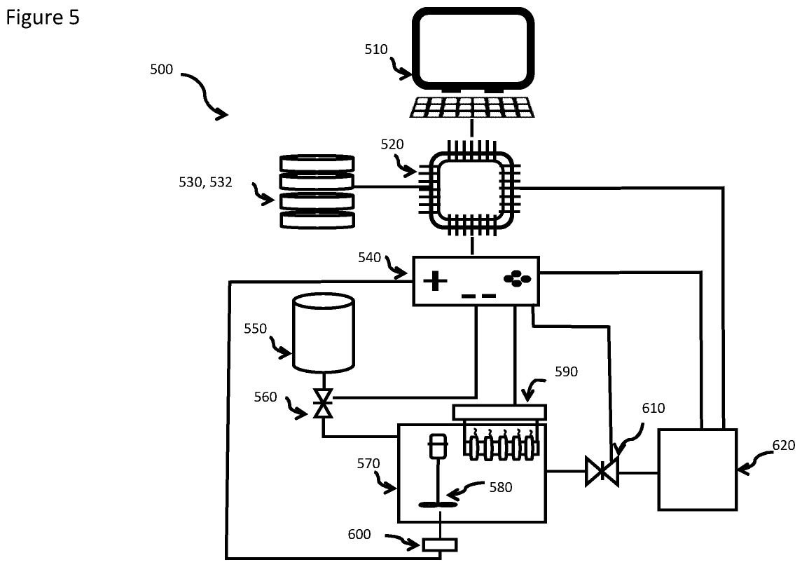

FIG. 5 shows an exemplary apparatus for controlling synthesis of a candidate material. An obtaining unit 510 is configured for receiving a digital representation of a material. In this example the obtaining unit is a client device communicatively coupled to a property determination unit 520. The obtaining unit may be communicatively coupled to the property determination unit by a bus system, wired, wirelessly as well as over via an internet protocol. A model unit 530, is configured to provide a data driven model trained based on based on digital representations of previously presented materials and at least two of their respective characteristic properties. In this example the model unit 530 comprises a database with the stored data driven model. The property determination unit 520 is configured to determine the at least two characteristic material properties of the candidate material based on the digital representation and the data driven model. A pareto unit 532 is configured to provide a provisional pareto front to the validation unit 522. In this example, the pareto unit comprises a database storing the provisional pareto front. The validation unit compares the preliminary praetor front with the determined at least two characteristic properties of the candidate material. Based on the comparison step a control file suitable for performing the synthesis is provided to a control unit 540. Control unit 540 is communicatively coupled to a providing unit 524 and is configured to control the synthesis equipment, 550, 560, 570, 580, 590. The control unit is further configured to receive the control file suitable for controlling synthesis. The synthesis equipment may comprise one or more reservoirs 550 for ingredients needed for the synthesis specification. And one or more valves 560 for regulating ingredient flow into reactor 570. In this example the reactor 570 may be equipped with a mixer 580 and a heating/cooling element 590. A motor 600 of the mixer may be controlled with the control unit 540. The control unit 540 controls the synthesis equipment according to the control file provided by the providing unit 524. Valve 610 may be controlled by the control unit to extract a sample from the reactor. The sample may then be measured in measurement apparatus 620. Measurement apparatus is configured to measure the at least two characteristic material properties of the synthesized candidate material. The measured dato may then be provided to the validation unit to determine whether the measured material characteristics are pareto optimal. In another example not shown, the synthesis and the measurements are performed in silico.

1. A computer implemented method for controlling synthesis of a material, in particular a polymer is proposed, the method comprising:
providing a digital representation associated with a synthesis specification of a candidate material
providing a data driven model trained based on digital representations of previously presented materials and at least two of their respective characteristic properties,
determining the at least two characteristic material properties of the candidate material based on the data driven model and the digital representation,
comparing the determined at least two characteristic material properties with the provisional pareto front,
based on the comparison providing a control file, suitable for controlling the synthesis of the candidate material.

2. The method according to claim 1, wherein the synthesis specification of the candidate material comprises a list of ingredients and machine-readable instructions for synthesizing material.

3. The method according to claim 1, wherein the digital representation is provided by a client device and the control file is received by a client device.

4. The method according to claim 1, wherein the provisional pareto front comprises a measure for uncertainty.

5. The method according to claim 1, wherein the at least two determined characteristic properties comprise a measure for uncertainty.

6. The method according to claim 1, wherein the comparing comprises determining if the determined characteristic material properties overlap with the provisional pareto front.

7. The method of claim 6, wherein the control file is provided if the comparing step determines an overlap.

8. The method of claim 1, wherein the method further comprises controlling the experiment, in particular by controlling flow rates of ingredients and reaction temperatures.

9. (canceled)

10. An apparatus for controlling synthesis of a material in particular a polymer is proposed, the apparatus comprising at least:
an obtaining unit configured to receive a digital representation of a candidate material,
a model unit configured to provide a data driven model trained based on digital representations of previously presented materials and at least two of their respective characteristic properties,
a pareto unit configured to provide a provisional pareto front associated with the at least two characteristic material properties for the subset of materials,
a property determination unit configured to determine the at least two characteristic material properties of the candidate material based on the data driven model and the digital representation,
a validation unit configured to compare the determined at least two characteristic material properties with the provisional pareto front,
a providing unit configured to, based on the comparison providing a control file, suitable for controlling the synthesis of the candidate material.

11. The apparatus of claim 10, communicatively coupled to a control unit for controlling the experiment.

12. A computer program product for controlling synthesis of a material, the computer program product comprising instructions, which, when executed on computing devices of a computing environment, is configured to carry out the steps of the method of controlling according to claim 1.

13. A computer implemented method for determining a provisional pareto front associated with characteristic material properties of materials in particular for screening comprising providing via a communication interface a set of materials, wherein each of the materials of the set of materials is described by their digital representation providing via the communication interface for of each of the materials of a subset of the set of materials at least two of their respective characteristic properties; providing a data driven model trained based on the digital representation of each of the materials of the subset of materials and at least two of their respective characteristic properties; predicting with the processing device the at least two characteristic material properties of remaining materials from the set of materials based on the data driven model; providing via the communication interface for each of the set of materials their at least two characteristic material properties; determining with the processing device from the set of materials a predicted pareto optimum for the at least two of the characteristic material properties; providing via the communication interface the determined provisional pareto front.

14. A computing apparatus including a processor and a memory storing instructions that, when executed by the processor, configure the apparatus to perform the method of claim 13.

15. A computer program product including instructions that, when processed by a computer, configure the computer to perform the method of claim 13.
